(19) United States
(12) Patent Application Publication
(10) Pub. No.: US 2022/0240980 A1
(43) Pub. Date: Aug. 4, 2022

(54) Endoscope Combined with Injection Needle

(71) Applicants: Chin-Piao CHANG, Miaoli County (TW); Hong-So KAO, Taipei City (TW)

(72) Inventors: Chin-Piao CHANG, Miaoli County (TW); Hong -So KAO, Taipei City (TW); HANS-JOACHIM KOORT, IN BONN (DE)

(21) Appl. No.: 17/165,960

(22) Filed: Feb. 3, 2021

(51) Int. Cl. A61B 17/34; A61B 1/018; A61B 1/06; A61B 1/00

(52) U.S. Cl. CPC A61B 17/3478; A61B 17/3421; A61B 1/00045; A61B 1/0684; A61B 1/00016; A61B 1/018

(57) ABSTRACT

The present invention provides an endoscope combined with an injection needle comprising an injection needle, an endoscope and a Y adapter. The endoscope comprises a cannula, a light source input and an output. The Y adapter has two branches, one branch has two interfaces to connect the injection needle and the light source input; and the other branch has an injection interface. The light source input is used to input an optical fiber beam and transmit the optical fiber beam to the cannula; and the output is connected to the extension part of the light source input. Therefore, the endoscope combined with an injection needle can use a disposable injection needle to replace a working channel, and the injection needle can be directly injected after being installed to the endoscope in the injection needle, so that the wound is small and no additional treatment is required after the operation.

### TECHNICAL FIELD OF THE INVENTION

The present invention relates to an endoscope combined with an injection needle, and more particularly to a working endoscope combined with the injection needle and the endoscope capable of injecting medicine directly after positioning the endoscope.

### DESCRIPTION OF THE PRIOR ART

Medical endoscopes and accessories thereof can enter the human body through various tubes for observation or as surgical equipment. It can be widely used in the human body's throat, stomach, intestines, gallbladder and other digestive systems and urinary systems. Further, the conventional endoscope inspection is performed through a lens installed at one end of a flexible tube or through a rigid tube. Different endoscopes are matched with different types of endoscope lenses; the tube of the endoscope enters the body, so that the light source enters in front of the tube and an observer can observe through the lens. Then the data from the endoscope is transmitted to the observer and related electronic equipment to help the doctor's diagnosis and treatment. However, when the endoscope with the rigid tube is used by the doctor, the endoscope enters the human body to find the source of disease. Then, when the doctor sees the source of disease to be injected, the doctor needs to fix the position of the endoscope before injecting, but the method may cause displacement problems.

Further, commercially available endoscopes and the working instruments are installed in the working channel. If the injection is to be carried out, the needle needs to be installed through the working channel, which is time-consuming and labor-intensive. Due to the large size of the working channel, it is necessary to make a larger opening on the skin and use the puncture cannula together. After the operation, it needs to be sutured and the recovery period is longer. Therefore, there is an urgent need for an endoscope combined with an injection needle that can overcome the above-mentioned problems.

In view of this, the inventor has invested a lot of research, development and effort, making breakthroughs and innovations, hoping to solve the current shortcomings with novel technical methods, not only bringing better products to the society, but also promoting industrial development at the same time.

### SUMMARY OF THE INVENTION

The main purpose of the present invention is to provide an endoscope combined with an injection needle; wherein a disposable injection needle is used to replace the working channel, and the endoscope is installed in the injection needle. When the endoscope enters the body, the injection site is observed to directly inject without positioning. Further, the wound is small, and the recovery period can be shortened without additional treatment after surgery. Furthermore, disposable injection needles can eliminate the need for disinfection and reduce the risk of infection.

To achieve the above objective, the present invention provides an endoscope combined with an injection needle comprising an injection needle, an endoscope and a Y adapter; wherein the outer diameter of the injection needle is 1.2 to 2.5 mm, the length of the injection needle is 40 to 450 mm and the front end of the injection needle has a first surface. Furthermore, the endoscope comprises a cannula, a light source input and an output. The cannula has a lens and a tube part, the tube part of the cannula is located in the injection needle, and the lens is arranged at the front end of the tube part to capture multiple images. Further, the light source input is used to input a light source and transmit the light source to the cannula; and the output is connected to the extension part of the light source input; and both ends of the Y adapter are respectively connected to the injection needle and the light source input.

In some embodiments, the injection needle further includes an optical component which is installed in front of the lens, and the optical component can be a conventional flat lens assembly.

In some embodiments, the optical component is a hollow round tip triangular structure composed of a parallel plate and a semi-cylinder with an inclined surface.

In some embodiments, the optical component is a solid round tip triangle structure, and a slope part of the solid round tip triangular structure has a stepped processing plane. Further, the stepped processing plane is partially or completely disposed on the slope part, and the stepped processing plane can be a micron-level structure or a nano-level structure.

In some embodiments, the first surface is a flat surface, an inclined surface, a conical surface, or a circular arc surface.

In some embodiments, the endoscope further comprises a switching mechanism connected to the output; wherein the switching mechanism is an optical fiber light collection structure, an image transmission switching mechanism or a power source required for an LED light source; and the switching mechanism includes a wireless transmission module or traditional wire transmission.

In some embodiments, the Y adapter has a first branch and a second branch; wherein the first branch has two interfaces to connect the injection needle and the light source input respectively.

In some embodiments, the second branch has an injection interface for injecting a medicine, the first branch connects to the second branch, and the medicine passes through the first branch to enter the injection needle, so that the medicine is distributed between the injection needle and the light source after the medicine is injected.

In some embodiments, the output is connected to an output device, an image reading device and an image processing device; wherein the output device comprises a communication device, a control device, and a display device. The communication device is coupled to the lens and is used for sending the images captured by the lens. Further, the control device is communicatively connected to the communication device to receive the images sent by the communication device, and send the images to the display device after processing. Further, the image processing device may include a signal amplifier or a noise remover. Furthermore, the display device is coupled to the control device to receive and show the images after processing; wherein the output is connected to the output device wirelessly, and the wireless connection includes Bluetooth, ZigBee, Wi-Fi or RF etc.

### BRIEF DESCRIPTION OF THE DRAWINGS

The above and other objectives, features and other advantages of the present invention will be more clearly understood from the following detailed description taken in conjunction with the accompanying drawings, in which:

FIG. 1 is an exploded view of a structure of an endoscope combined with an injection needle according to the present invention;

FIG. 2 is a schematic view of a structure of the endoscope combined with the injection needle according to the present invention;

FIG. 3A is an enlarged side perspective view of A in FIG. 1 according to the present invention;

FIG. 3B is one enlarged side perspective view of A in FIG. 1 according to the present invention;

FIG. 3C is another enlarged side perspective view of A in FIG. 1 according to the present invention;

FIG. 3D is the other enlarged side perspective view of A in FIG. 1 according to the present invention;

FIG. 4A to 4C are schematic views of different types of optical fiber beams surrounding the lens needle according to the present invention;

FIG. 5 is a partial cross-sectional enlarged view of the injection needle according to the present invention; and

FIG. 6 is a schematic view of an image output of the endoscope according to the present invention.

### DETAILED DESCRIPTION OF THE PREFERRED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

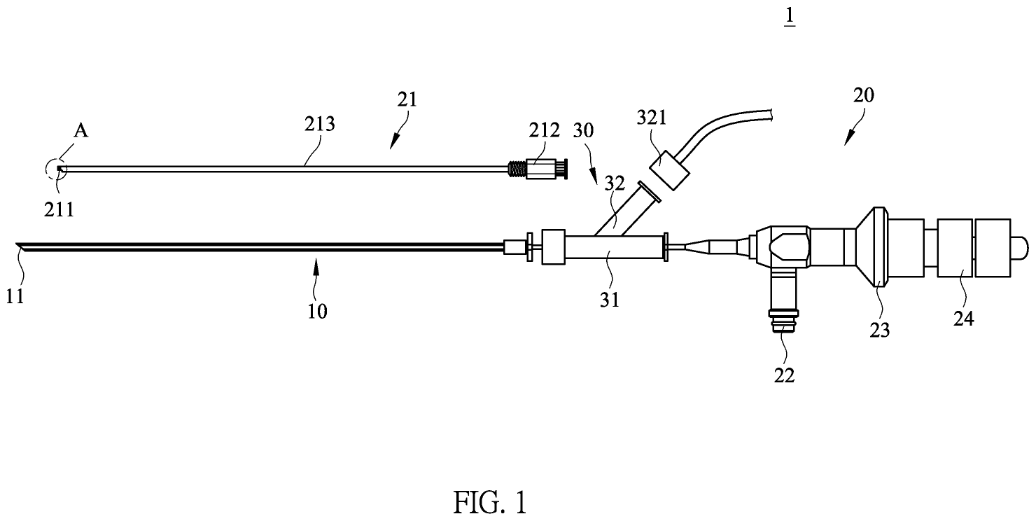

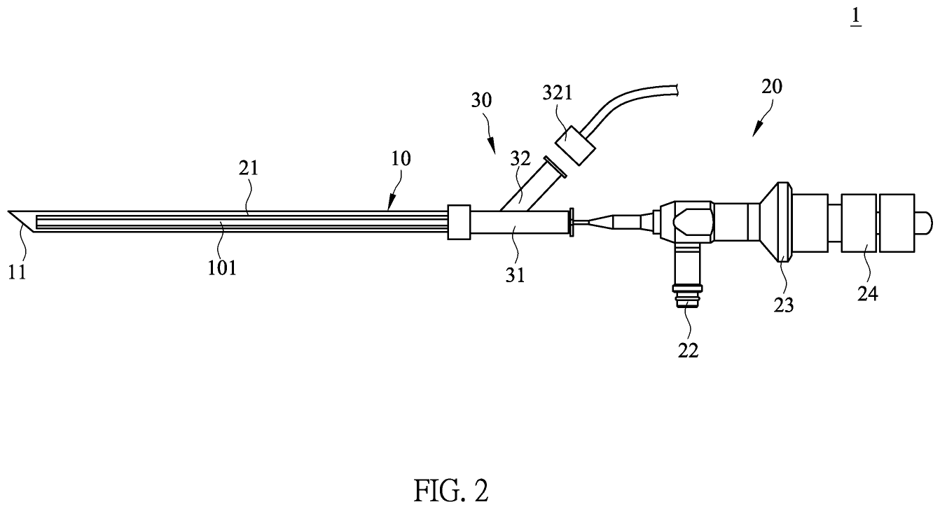

Please refer to FIG. 1 to FIG. 2, FIG. 1 is an exploded view of a structure of an endoscope combined with an injection needle according to the present invention; and FIG. 2 is a schematic view of a structure of the endoscope combined with the injection needle according to the present invention.

As shown in FIG. 1 to FIG. 2, the present invention provides an endoscope 1 combined with an injection needle comprising an injection needle 10, an endoscope 20 and a Y adapter 30; wherein the injection needle 10 is made of a stainless steel tube and has an outer diameter of 1.2 to 2.5 mm, and the front end of the injection needle 10 has a first surface 11, and the style of the first surface 11 can be randomly varied based on the user's requirements and preferences. In the present Embodiment, the first surface 11 is an inclined surface and can be used for puncturing. The length of the injection needle 10 is 40 to 450 mm, preferably 60 to 300 mm. The injection needle 10 is a disposable injection needle, which does not need to be sterilized and can reduce the risk of infection.

Secondly, the endoscope 20 comprises a cannula 21, a light source input 22, an output 23 and a switching mechanism 24; wherein the cannula 21 has a lens 211, a head part 212 and a tube part 213, and the lens 211 is arranged at the front end of the tube part 213 of the cannula 21 to capture images; wherein the cannula 21 can be movable or fixed inside the injection needle 10. Moreover, the light source input 22 is used to input a light source 101 and transmit the light source 101 to the tube part 213. The light source 101 can be an optical fiber beam or an LED light source. When the light source 101 is the LED light source, the light source is located at the front end of the tube part 213, and LED supply wire inside the tube part 213. In the present Embodiment, the light source 101 is the optical fiber beam, and the light source input 22 is used to input the optical fiber beam and transmit the optical fiber beam to the cannula 21. Further, the output 23 is connected to the extension part of the light source input 22 and the switching mechanism 24. Moreover, the switching mechanism 24 is an optical fiber light collection structure, an image transmission switching mechanism or a power source required for an LED light source; and the switching mechanism 24 includes a wireless transmission module or traditional wire transmission.

In addition, the Y adapter 30 is respectively connected to the injection needle 10 and the light source input 22; wherein the Y adapter 30 has a first branch 31 and a second branch 32. The first branch 31 has two interfaces to connect the injection needle 10 and the light source input 22 respectively; wherein the head part 212 of the cannula 21 is installed in the first branch 31; the tube part 213 is installed in the injection needle 10; and the optical fiber beam from the light source input 22 enters the first branch 31 and then is transmitted into the tube part 213. Furthermore, the second branch 32 has an injection interface 321 for injecting a medicine. The second branch has an injection interface for injecting a medicine, the first branch connects to the second branch, and the medicine passes through the first branch to enter the injection needle, so that the medicine is distributed between the injection needle and the light source after the medicine is injected.

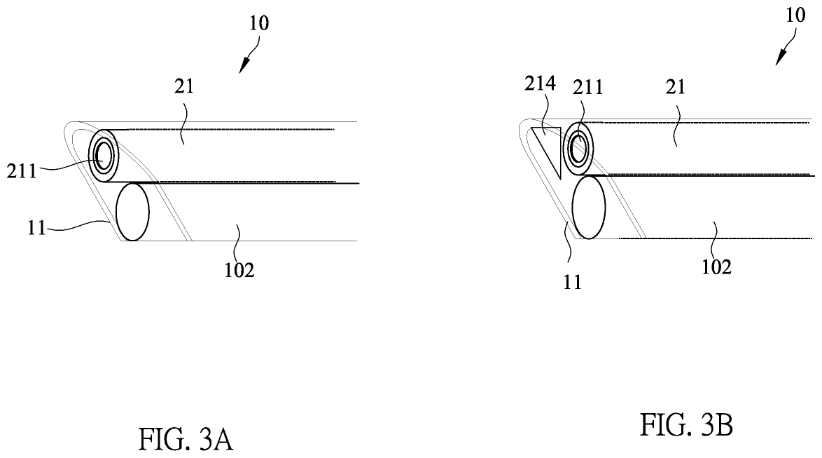

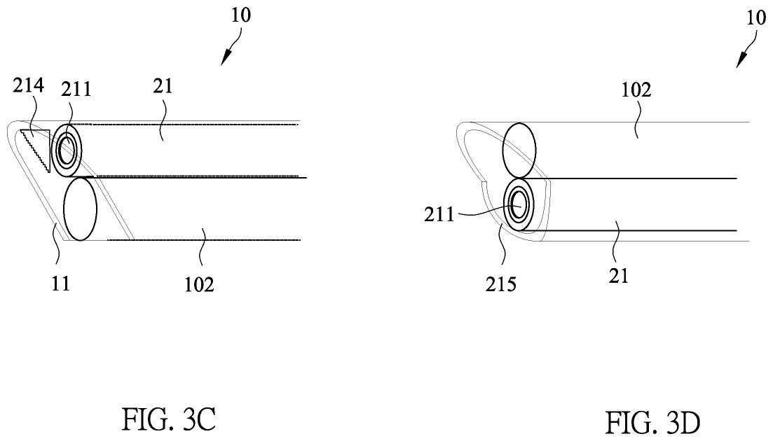

Please refer to FIG. 3A to FIG. 3D, FIG. 3A is an enlarged side perspective view of A in FIG. 1 according to the present invention; FIG. 3B is one enlarged side perspective view of A in FIG. 1 according to the present invention; FIG. 3C is another enlarged side perspective view of A in FIG. 1 according to the present invention; and FIG. 3D is the other enlarged side perspective view of A in FIG. 1 according to the present invention.

As shown in FIG. 3A, the lens 211 is arranged at the front end of the tube part 213 of the cannula 21 to capture images; wherein the lens 211 can be a CCD (Charge Coupled Device) lens or a CMOS (Complementary Metal-Oxide Semiconductor or a complementary metal oxide semiconductor) lens. In addition, the injection needle 10 further comprises a solid part 102. Before the injection needle 10 enters the body, the solid part 102 is movably arranged in the injection needle 10 first, and the solid part 102 can prevent the body tissue from entering the injection needle 10. After the injection needle 10 enters the body, the solid part 102 is taken out, and the medicine is injected so that the medicine can be distributed in the injection needle 10. As shown in FIG. 3B, the structure of FIG. 3B is substantially the same as the above FIG. 3A, but the differences are as follows. First, the injection needle 10 of FIG. 3B further includes an optical component 214, and the optical component 214 is installed in front of the lens 211. Secondly, the optical component 214 is a hollow round tip triangular structure composed of a parallel plate and a semi-cylinder with an inclined surface, and the optical component 214 can be a glass or an optical plastic. The optical component 214 can avoid the problem of optical refraction of the image captured by the lens 211. As shown in FIG. 3C, the structure of FIG. 3C is substantially the same as the above FIG. 3B, but the differences are as follows. First, the optical component 214 of FIG. 3C is a solid round tip triangular structure, and a slope part of the solid round tip triangular structure has a stepped processing plane. Further, the stepped processing plane is partially or completely disposed on the slope part, and the stepped processing plane can be a micron-level structure or a nano-level structure. As shown in FIG. 3D, the structure of FIG. 3D is substantially the same as the above FIG. 3A, but the difference is that the position of the lens 211 is installed in the injection needle. The lens 211 of FIG. 3A is installed above the injection needle 10, but the lens 211 of FIG. 3D is installed under the injection needle 10 and the front end of the cannula 21 has a second surface 215 which is a circular arc surface or a conical surface to avoid the problem of optical refraction. Moreover, the lens 211 can be fixed in the cannula 21, or the lens 211 can be movably arranged in the cannula 21.

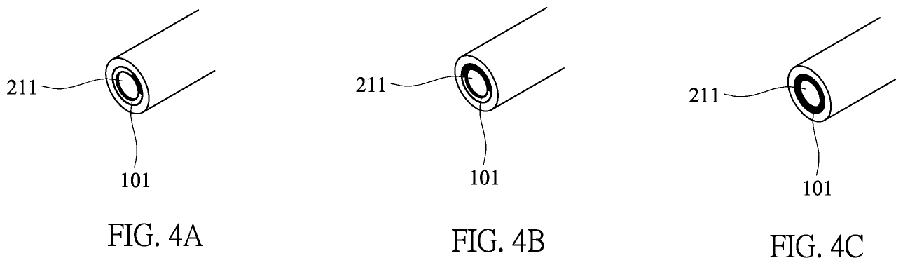

Please refer to FIG. 4A to FIG. 4C, FIG. 4A to 4C are schematic views of different types of optical fiber beams surrounding the lens needle according to the present invention.

As shown in FIG. 4A to FIG. 4B, the light source 101, such as an optical fiber beam, surrounds the periphery of the lens 211, and the light source 101 may partially surround the periphery of the lens 211; or as shown in FIG. 4C, the light source 101 may completely surround the periphery of the lens 211. Secondly, a chip can be arranged on the lens 211 and the light source 101 is surrounded the chip, and the chip can be used to transmit the image to the output.

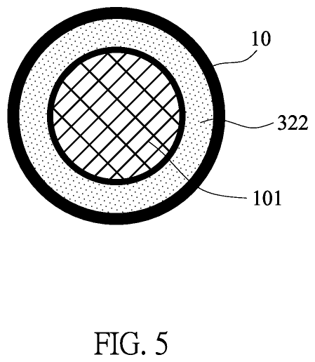

Please refer to FIG. 5, FIG. 5 is a partial cross-sectional enlarged view of the endoscope combined with the injection needle according to the present invention.

As shown in FIG. 1 and FIG. 5, the second branch 32 has the injection interface 321 for injecting a medicine 322. The medicine 322 is distributed between the injection needle 10 and the light source 101 after the medicine 322 is injected; and the injection needle 10 dads the medicine 322 and the light source 101.

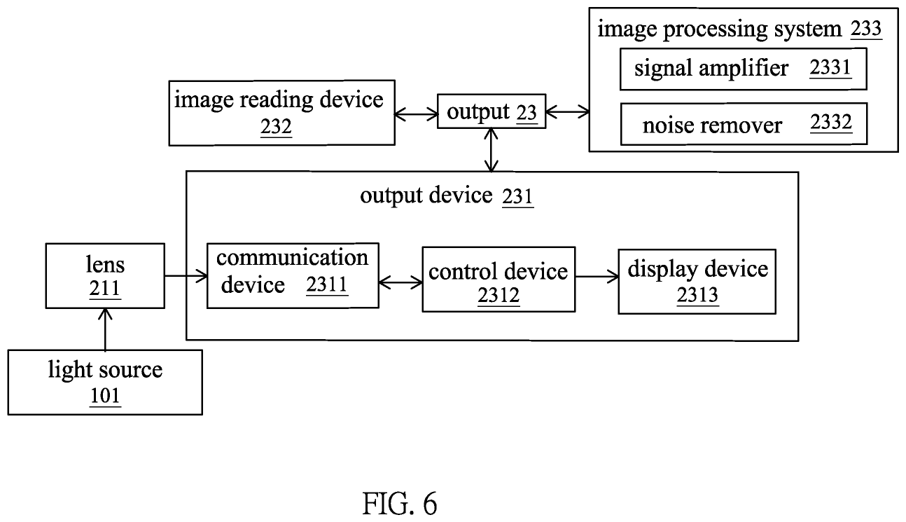

Please refer to FIG. 6, FIG. 6 is a schematic view of an image output of the endoscope needle according to the present invention.

As shown in FIG. 6, the output 23 can be an eyepiece, or the output 23 can be connected to an output device 231, an image reading device 232 and an image processing system 233; wherein the image processing device 233 may include a signal amplifier 2331 or a noise remover 2332. The output device 231 comprises a communication device 2311, a control device 2312, and a display device 2313. The communication device 2311 is coupled to the lens 211 and is used for sending the images captured by the lens 211. Further, the control device 2312 is a CPU, and it is communicatively connected to the communication device 2311 to receive the images sent by the communication device 2311, and send the images to the display device 2313 after processing. Furthermore, the display device 2313 is coupled to the control device 2312 to receive and show the images after processing, and a doctor can observe the body through the lens 211, and send the data back to the communication device 2311 for further image processing and display the result on the display device 2313 to facilitate the doctor's diagnosis and treatment. In one embodiment, the output 23 can be connected to the output device 231 wirelessly, and the wireless connection can include Bluetooth, ZigBee, Wi-Fi or RF, but the invention is not limited thereto. Secondly, the output device 231 can be a display screen, a portable electronic device or a VR glasses. The doctor can watch the image captured by the lens by wearing the VR glasses, but the creation is not limited thereto. Furthermore, the image reading device 232 is coupled to the lens 211 to read the images captured by the lens 211; and the image processing device 233 can process the images read by the image reading device 232.

The foregoing description, for the purpose of explanation, has been described with reference to specific embodiments. However, the embodiments were chosen and described in order to best explain the principles of the disclosure and its practical applications, to thereby enable others skilled in the art to best utilize the disclosure and various embodiments with various modifications as are suited to the particular use contemplated. The embodiments depicted above and the appended drawings are exemplary and are not intended to be exhaustive or to limit the scope of the disclosure to the precise forms disclosed. Modifications and variations are possible in view of the above teachings.

I claim:

1. An endoscope combined with an injection needle, comprising:
an injection needle having an outer diameter of 1.2 to 2.5 mm and a length of 40 to 450 mm, the front end of the injection needle having a first surface;
an endoscope comprising:
a cannula having a lens and a tube part, the tube part of the cannula located in the injection needle, and the lens arranged at the front end of the tube part to capture multiple images;
a light source input used to input a light source and transmit the light source to the tube part; and
an output connected to the light source input; and
a Y adapter having two interfaces respectively connected to the injection needle and the light source input.

2. The endoscope combined with an injection needle mentioned in claim 1, wherein the injection needle further includes an optical component which is installed in front of the lens.

3. The endoscope combined with an injection needle mentioned in claim 2, wherein the optical component is a hollow round tip triangular structure composed of a parallel plate and a semi-cylinder with an inclined surface.

4. The endoscope combined with an injection needle mentioned in claim 2, wherein the optical component is a solid round tip triangle structure, a slope part of the solid round tip triangular structure has a stepped processing plane, the stepped processing plane is partially or completely disposed on the slope part, and the stepped processing plane can be a micron-level structure or a nano-level structure.

5. The endoscope combined with an injection needle mentioned in claim 1, wherein and the first surface is a flat surface, an inclined surface, a conical surface, or a circular arc surface.

6. The endoscope combined with an injection needle mentioned in claim 1, wherein the endoscope further comprises a switching mechanism connected to the output; the switching mechanism is an optical fiber light collection structure, an image transmission switching mechanism or a power source required for an LED light source; and the switching mechanism includes a wireless transmission module or traditional wire transmission.

7. The endoscope combined with an injection needle mentioned in claim 1, wherein the Y adapter has a first branch and a second branch, and the first branch has two interfaces to connect the injection needle and the light source input respectively.

8. The endoscope combined with an injection needle mentioned in claim 7, wherein the second branch has an injection interface for injecting a medicine, and the medicine is distributed between the injection needle and the optical fiber beam after the medicine is injected.

9. The endoscope combined with an injection needle mentioned in claim 1, wherein the output is connected to an output device, an image reading device or an image processing device; the output device comprises a communication device, a control device, and a display device; the communication device is coupled to the lens and is used for sending the images captured by the lens; the control device is communicatively connected to the communication device to receive the images sent by the communication device; the images are sent to the display device after processing; and the display device is coupled to the control device to receive and show the images after processing.

10. The endoscope combined with an injection needle mentioned in

9, wherein the output is connected to the output device wirelessly, and the wireless connection includes Bluetooth, ZigBee, Wi-Fi or RF.
